(19) United States
(12) Patent Application Publication
(10) Pub. No.: US 2008/0214656 A1
(43) Pub. Date: Sep. 4, 2008

(54) Compounds With Diphenoyl-Structure For the Treatment of Immune Diseases

(75) Inventors: Jong Soon Lim, Daejeon (KR); Sang Ik Lee, Daejeon (KR)

Correspondence Address: EDWARDS ANGELL PALMER & DODGE LLP, P.O. BOX 55874, BOSTON, MA, 02205, US

(73) Assignee: Neurogenex Co., Ltd., Seoul (KR)

(21) Appl. No.: 11/661,153

(22) PCT Filed: Aug. 22, 2005
(86) PCT No.: PCT/KR05/02764

§ 371 Date: Nov. 28, 2007

(30) Foreign Application Priority Data
Aug. 23, 2004 (KR) 10-2004-0066463

(51) Int. Cl. A61K 31/235; A61K 31/37; A61P 3/00; A61P 19/00

(52) U.S. Cl. 514/450; 514/533

(57) ABSTRACT

A composition comprising compounds with a diphenoyl (DP 5 structure for preventing or treating an immune disease and a prophylactic or therapeutic method for the immune disease based on the application of the compounds are provided. The compounds with the DP structure increase the number and activity of regulatory T cells involved in regulating an accelerated immune system. Also, the compounds can be an effective prophylactic or therapeutic agent for various immune diseases including transplantation rejection, graft-versus-host diseases, autoimmune diseases, and hypersensitive inflammatory diseases.

### FIELD OF THE INVENTION

The present invention relates to a composition comprising compounds with a diphenoyl structure as an effective component for the prevention or treatment of immune diseases.

### DESCRIPTION OF THE RELATED ART

Immunity is an adaptive internal defense system of a living body against foreign biological materials. The immune system has been developed together with surveillance and defense mechanisms that recognize and remove pathogenic foreign microorganisms such as bacteria and viruses. Therefore, the living body identifies its own cells or tissues (with self antigens) from foreign microorganisms (with non-self antigens). The immune system does not respond to the self-antigens, or does not exhibit immune function even if it responds to the self-antigens. This phenomenon is called immune tolerance.

In a series of immune responses, when abnormal responses occur, lymphocytes (especially T cells) strongly respond to self-antigens and may damage tissues. This abnormal attack of self-tissues is called an autoimmune disease. Sometimes, autoimmune diseases occur when a normal immune response to bacteria or viruses is changed to an immune response to a self-antigen. Tissue damage or infection induced by autoimmune diseases results in various types of inflammation.

Inflammation is an immune response caused by tissue damage, infection by microorganisms, allergens and so forth. Typically, heat, swelling, redness and pain are four signs of inflammation. Also, inflammation can be classified into an acute inflammation and a chronic inflammation. Specifically, the chronic inflammation is considered as a main factor of various immune related diseases including autoimmune diseases.

In the case of organ transplantation or bone marrow (hematopoietic stem cells) transplantation, a recipient shows an immune rejection response against transplanted tissues. Even if such immune rejection is a normal immune response, suppression of the immune rejection is preferable in view of treatment.

Currently used immune suppressants can be classified into proliferation inhibitors, anti-inflammatory steroids, signal transduction inhibitors, and monoclonal antibodies, which have been introduced recently. However, as described in an article by Miller (Semin.Vet.Med.Surg., 12(3), pp. 144-149, 1997), those proliferation inhibitors such as methotrexate and cyclophosphamide are strongly effective in inhibiting cell growth but severely toxic to rapidly-dividing normal cells of bone marrow and gastrointestinal mucous membranes. Also, they may frequently cause hepatic dysfunction. Anti-inflammatory steroids such as dexamethasone and prednisolone used to induce immune suppression may have adverse effects including infection, abnormal metabolism, high blood pressure, and diabetes, and a frequent dosage of such anti-inflammatory steroids gives drug tolerance. According to an article by Liu et al. (Cell, 66, pp. 807-815, 1991) and another article by Henderson et al.(Immun., 73, pp. 316-321, 1991), drugs like cyclosporine, FK506 used to activate lymphocytes and inhibit continuous proliferation of cells inhibit calcineurin, which is a calcium-dependent phosphatase, thereby inhibiting transduction of an activation signal. These drugs are most commonly used immune suppressants and often used to prevent or treat a transplantation rejection response when those organs such as kidney, liver, heart, bone marrow, and so on are transplanted. These drugs are used for the therapeutic purpose for various chronic inflammatory diseases including inflammatory bowel diseases, psoriasis, rheumatoid arthritis, panmyelophthisis, and multiple sclerosis or autoimmune related immune diseases. However, those drugs like cyclosporin have a narrow dosage range and severe toxicity including nephrotoxicity, hepatotoxicity, hypertension, cancer, and neurotoxicity. These various toxicities arising when using the aforementioned drugs are described in an article by Philip and Gerson (Clin. Lab. Med., 18(4), pp. 755-765, 1998) and in an article by Hojo et al. (Nature, 397, pp. 530-534, 1999).

The aforementioned immune suppressants forcefully lower the accelerated activation of immunocytes through an externally dosed drug without considering regulatory functions in the living body. However, recent studies have introduced regulatory T cells (Treg), which can actively regulate activities of other T cells that may damage tissues, and control the accelerated immune system to control autoimmune diseases or chronic inflammatory diseases.

Among numerous T lymphocytes, a subset of T cells called CD4⁺CD25⁺ is a regulatory T cells. For instance, according to Salomon et al. (Immunity, 12(4), pp. 431-440, 2000), Read et al. (J. Exp. Med., 192(2), pp. 295-302, 2000) and Annacker et al. (J. Immunol., 166(5), pp. 3008-3018, 2001), the regulatory T cell subset in an experiment on rodents can inhibit autoimmune diabetes, prevent inflammatory bowel diseases, and inhibits proliferation and activation of other pathogenic T cells. Particularly, according to Read and Powrie (Curr. Opin. Immunol., 13(6), pp. 644-649, 2001), several mechanisms of action of the regulatory T cells have been proposed, including inhibition by direct contact with other immunocytes through expressed inhibitory molecules at the surfaces of regulatory T cells, or inhibition by secreting transforming growth factor-beta (TGF-β), which is known as one of the important immune-suppressive cytokine.

Foxp3 which encodes a transcription factor is the most specific marker that can distinguish the regulatory T cells from other T cells (Fontenot et al., Nat. Immunol., 4, pp. 330-336, 2003). Foxp3 participates in regulating immune responses by binding around a promoter region of a cytokine gene related to activation of lymphocytes like interleukin-2 (Schubert et al., J. Biol. Chem., 267, pp. 37672-37679, 2001). Khattri et al. (Nat. Immunol., 4, pp. 337-342, 2003) have reported that murine CD4⁺CD25⁻ T cells transduced with Foxp3 gene, showed immune suppression. In contrast, a mouse without Foxp3 has developed lymphoproliferative diseases similar to autoimmune diseases. Also, overexpression of Foxp3 in mice with immune-regulatory deficiency, delayed the onset of autoimmune diseases (Fontenot et al., Nat. Immunol., 4, pp. 330-336, 2003).

The above experimental results reveal that excessive activation of immune cells, which is a cause of abnormal immune diseases, is resulted from absence or hypofunction of regulatory T cells. And they also indicate that immune diseases can be controlled by using regulatory functions pertained to human bodies. This new method is different from methods that typical immune suppressants employ. Recovery or increase of the regulatory T cell activation can be estimated by the level of Foxp3 expression and thus it is possible to screen new immune regulatory agents that can increase the activation of regulatory T cells involved in the regulation of immune system.

A diphenoyl (DP) structure, especially a hexahydroxydiphenoyl (HHDP) structure, is commonly discovered in various plant compositions such as chebulagic acid separated from medicinal plants such as Terminalia chebula or Erodium stephanianum Willd, punicalagin separated from Punica granatum, which is an eatable and medicinal plant, and corilagin separated from Acer nikoense, which is an ornamental and medicinal plant. Although there have been lots of researches on various biological activities of such plant compositions with the HHDP structure, it has not been reported yet that compounds with the HHDP structure recover or increase the activation of regulatory T cells, thereby preventing or treating immune diseases.

### DETAILED DESCRIPTION OF THE INVENTION

Substances from the natural products have been a focus of our research in the development of immune regulatory agents to control various immune diseases. Compounds including a diphenoyl (DP) structure, preferably a hexahydroxydiphenoyl (HHDP) structure, enhance activation of regulatory T cells, which are considered as a key to the control of autoimmune diseases and hypersensitive inflammatory diseases. Among compounds including the DP structure, preferably the HHDP structure, chebulagic acid and punicalagin show a strong immune suppressive activities in a mixed leukocytes reaction in vitro. In in vivo test of chebulagic acid, more specifically, in murine model of rheumatoid arthritis, which is an autoimmune disease, chebulagic acid is capable of suppressing the onset and progression of a chronic inflammatory autoimmune disease by regulating the immune system. In murine model of asthma, which is a hypersensitive inflammatory disease, chebulagic acid is also capable of regulating the immune system, thereby inhibiting inflammation. These effects are caused by increasing the number and activation of regulatory T cells. Our results demonstrate that compounds including the DP structure, particularly the HHDP structure, enhance the activation of regulatory T cells to thereby control autoimmune diseases and hypersensitive inflammatory diseases.

It is, therefore, an objective of the present invention to provide a composition including compounds with a DP structure, defined by the chemical formula 1 provided below as an effective component for preventing or treating immune disease.

It is another objective of the present invention to provide a method for preventing or treating immune disease by administering a composition including compounds with a DP structure, defined by the chemical formula 1 provided below as an effective component to patients, wherein the immune disease includes an organ transplantation rejection response, a graft-versus-host disease, an autoimmune disease, and a hypersensitive inflammatory disease.

It is a further objective of the present invention to provide a use of compounds with a DP structure, defined by the chemical formula 1 provided below, to prevent or treat an immune disease including an organ transplantation rejection response, a graft-versus-host disease, an autoimmune disease, and a hypersensitive inflammatory disease.

According to an aspect of the present invention, there is provided a composition including compounds with a DP structure defined by the chemical formula 1 as an effective component for preventing or treating an immune disease. The chemical formula 1 is defined as follows.

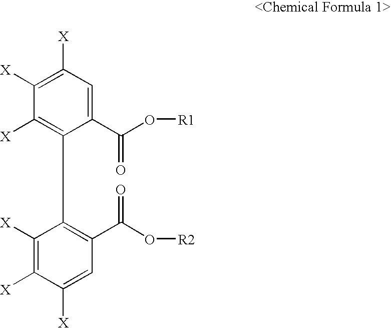

In the above chemical formula 1, X is selected from the group consisting of hydrogen (H), hydroxy (OH), halogen, cyano (CN), nitro (NO), amine (NH₂), sulfonyl (SO₂), methyl (CH₃), low-grade alkoxy, and low-grade alkyl. Particularly, the halogen is selected from the group consisting of fluorine (F), chlorine (Cl), bromine (Br), and iodine (I). Also, the above X is not necessarily the same molecule.

R1 and R2 or O—R1 and O—R2 are selected from the group consisting of H, OH, halogen, CN, NO, NH₂, SO₂, CH₃, alkoxy, alkyl, alkenyl, alkinyl, aryl, heterocycle, and cycloalkyl. Particularly, the halogen is selected from the group consisting of F, Cl, Br, and I. The above chemical structure can simultaneously bind with a saccharide compound or heterocycle group at the R1 and R2 sites. Also, the above chemical structure can also make covalent bonds with amino acids, peptides, proteins, and nucleic acids at the R1 and R2 sites.

Preferably, the compounds with the DP structure contain a hexahydroxydiphenoyl HHDP structure defined as below.

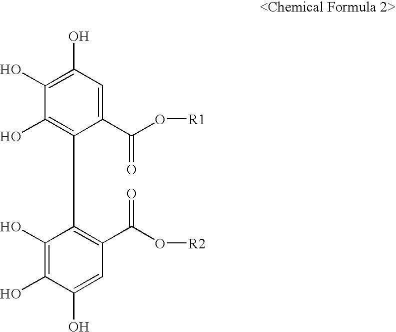

In the above chemical formula 2, R1 and R2 or O—R1 and O—R2 are selected from the group consisting of H, OH, halogen, CN, NO, NH₂, SO₂, CH₃, alkoxy, alkyl, alkenyl, alkinyl, aryl, heterocycle, and cycloalkyl. Particularly, the halogen is selected from the group consisting of F, Cl, Br, and I. The above chemical structure can simultaneously bind with a saccharide compound or heterocycle group at the R1 and R2 sites. Also, the above chemical structure can also make covalent bonds with amino acids, peptides, proteins, and nucleic acids at the R1 and R2 sites.

The compounds including the HHDP structure defined by the above chemical formula 2 can be produced from natural substances through the known extraction method or can be synthesized by employing the known method in the art.

Examples of such compounds are chebulagic acid separated from a medicinal plant such as Terminalia chebula or Erodium stephanianum Willd, punicalagin separated from Punica granatum, which is an eatable and medicinal plant, corilagin separated from Acer nikoense, which is an ornamental and medicinal plant, and Pedunculagin. However, the compounds including the HHDP structure defined by the above chemical formula 2 produced from natural substances or synthesized, are not limited only to these exemplary substances. The aforementioned chebulagic acid is defined as follows.

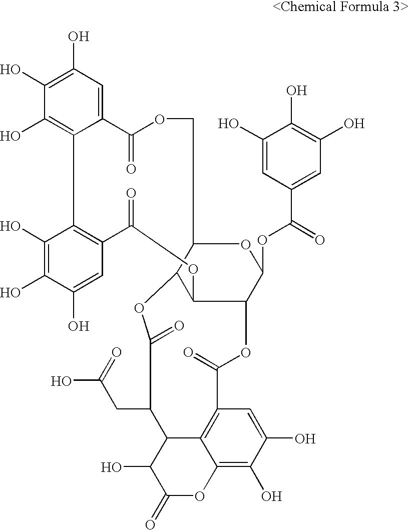

Punicalagin is defined as the following chemical formula.

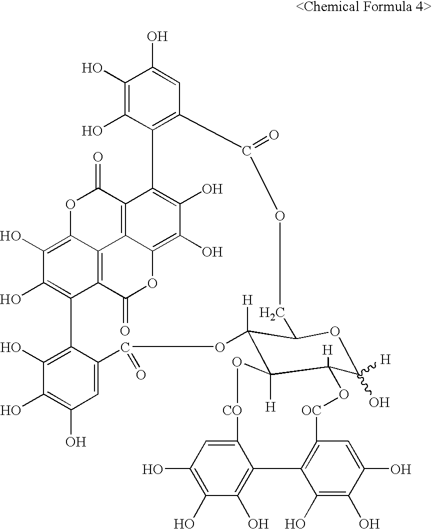

The aforementioned corilagin is defined as the following chemical formula.

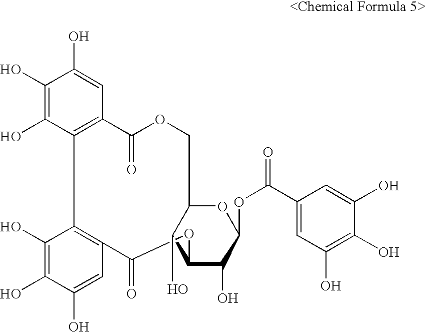

The aforementioned Pedunculagin is defined as follows.

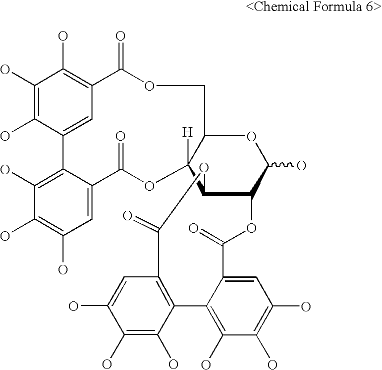

In addition to the aforementioned compounds with the purely separated HHDP structure defined as the chemical formulas 3 to 6, extracts including chebulagic acid separated from plants that can separate the compounds, for instance, plant extracts of Terminalia Chebula or Brodium stephanianum Willd can be used for the above described composition for treating various immune diseases.

The term “immune disease” is an immune response that is caused by an external source or a self-antigen and is not beneficial to a living body. An inflammatory response can be induced due to the immune response caused by the external source or the self-antigen, and such inflammatory response can be classified into chronic inflammation and acute inflammation. Particularly, chronic inflammation is closely related to autoimmune diseases such as rheumatoid arthritis, psoriasis and inflammatory bowel diseases, asthma, atherosclerosis, and Alzheimer's disease. (Balkwill and Mantovani, Lancet, 357(9255), pp. 539-545, 2001).

Although organ/tissue transplantation rejection and graft-versus-host diseases are normal immune responses, it is preferable to suppress the normal immune response to harmonize a transplanted or grafted organ/tissue with a living body. It is exemplified in the embodiments of the present invention that the immune suppression can be achieved by activating relevant cells that can suppress the immune response, especially the regulatory T cells, instead of direct suppression of an immune response via a drug.

As verified in the embodiments of the present invention, the composition including the DP structure, particularly the HHDP structure for suppressing an immune response, increases the number of regulatory T cells and enhances activation of the regulatory T cells. Also, the composition inhibits proliferation of lymphocytes caused by recognition of allogenic leukocytes. Further, the composition can delay or suppress the onset of immune diseases in various animal models with rheumatoid arthritis, asthma and organ transplantation, and treat the immune diseases.

Hence, the composition for treating immune diseases can preferably prevent or treat undesired immune diseases by activating regulatory T cells. Examples of the undesired immune diseases that can be prevented or treated by the above composition are an organ transplantation rejection response (Kingsley et al. (J. Immunol., 168, pp. 1080-1086, 2002), a graft-versus-host disease (Hequet et al. Am. J. Transplant., 4(6), pp. 872-878, 2004), and an autoimmune disease and a hypersensitive inflammatory disease (Thompson and Powrie, Curr. Opin. Pharmacol., 4(4), PP. 408-414, 2004).

Examples of the autoimmune disease can be the aforementioned rheumatoid arthritis (Morgan et al., Arthritis Rheum., 48(5), pp. 1452-1460, 2003), psoriasis, inflammatory bowel diseases (Martin et al., J. Immunol., 172(6), pp. 3391-3398, 2004), diabetes mellitus (Peng et al., Proc. Natl. Acad. Sci. USA, 101(13), pp. 4572-4577, 2004), ulcerative colitis (Powrie et al., Norvatis Found. Symp., 252, pp. 92-98, 2003), multiple sclerosis (Adorini, J. Neurol. Sci., 223(1), pp. 13-24, 2004), dermatosclerosis, myasthenia gravis, polymyositis, dermatomyositis, autoimmune hemolytic anemia, vasculitis syndrome, and systemic erythematosus lupus (Crispin et al., J. Autoimmun., 21(3), pp. 273-276, 2003). Examples of the hypersensitive inflammatory disease are asthma and allergies (Rhbinson and Dao, J. Allergy Clin. Immunol., 114(2), pp. 296-301, 2004).

According to another aspect of the present invention, there is provided a method for preventing or treating an immune disease by administering a composition including compounds with a DP structure defined as the chemical formula 1 to those patients who need to control immune regulatory actions.

Preferably, the method regulates an immune response by activating regulatory T cells involved in the immune regulatory actions. More preferably, exemplary embodiments provide a method for preventing or treating an organ transplantation rejection response, a graft-versus-host disease, an autoimmune disease, or a hypersensitive inflammatory disease.

According to a further aspect of the present invention, there are provided compounds with a DP structure defined as the chemical formula 1, in which the compounds can be used to prevent or treat an immune disease, including an organ transplantation rejection response, a graft-versus-host disease, an autoimmune disease, and a hypersensitive inflammatory disease.

The composition of the present invention can be formulated in oral types, including powders, granules, tablets, capsules, suspensions, emulsions, syrups, and aerosols, sterile parenteral types, non-oral types like ointments, and suppositories. Examples of carriers, excipients and diluents include lactose, dextrose, sucrose, sorbitol, mannitol, xylitol, erythritol, malitol, starch, rubber from Acacia, alginate, gelatin, calcium phosphate, calcium silicate, cellulose, methyl cellulose, microcrystalline cellulose, polyvinyl pirrolidone, water, methylhydroxybenzoate, propylhydroxybenzoate, talc, magnesium stearate, and mineral oils.

Solid preparations for an oral administration include tablets, pills, powders, granules, and capsules. The aforementioned solid preparation is mixed with at least one excipients selected from the group consisting of starch, calcium carbonate, sucrose, lactose, and gelatin. In addition to simple excipients, lubricants such as magnesium stearate and talc are used. Liquid preparations for the oral dose include suspensions, solutions, emulsions, and syrups. In addition to liquid paraffin and water, a commonly used simple dilution agent, the liquid type packaging material can include various excipients, such as wetting agents, sweetening agents, flavoring agents, and preservatives.

Those preparations for a non-oral administration include sterile liquid solutions, non-aqueous solvents, suspensions, emulsions, freezing drying agents, and suppositories. The non-aqueous solvents and suspensions include vegetable oils, such as propylene glycol, polyethylene glycol and olive oil and injectable ester, such as ethyloleate. Base preparations for the injection can include conventional additives such as solvents, isotonic agents, suspension agents, emulsion agents, stabilizers, and preservatives.

The composition according to the embodiments of the present invention can be administered through various methods including oral types, intravenous types, subcutaneous types, intracutaneous types, nasal types, intraperitoneal types, intramuscular types, and transdermal types. A dose of the composition can be varied depending on age, sex and weight of a patient and can be decided easily by those skilled in the art. For instance, approximately 50 mg of the composition per weight in kilograms can be administered for everyday or every other day, or can be administered one to three times for one day.

A dose of the composition can be increased or decreased depending on an administration pathway, a degree of a disease, sex, weight, and age. Therefore, it can be concluded that the aforementioned dose does not limit the scope of the present invention.

### BRIEF DESCRIPTION OF THE DRAWINGS

FIG. 1 is a graph illustrating that expression of Foxp3, which is a specific transcription factor of immune regulatory cells, is increased by compounds with a DP or HHDP structure, in which the compounds include chebulagic acid, punicalagin and corilagin representatively, and CT, CRN, CHE, and PCG express a control group, a corilagin treatment group, a chebulagic acid treatment group, and a punicalagin treatment group, respectively;

FIG. 2 is a graph illustrating that compounds with a DP or HHDP structure, particularly chebulagic acid and punicalagin, inhibit proliferation of lymphocytes in a mixed leukocyte reaction, in which CsA, CT, CHE, and PCG represent a cyclosporin A treatment group, a control group, a chebulagic acid treatment group, and a punicalagin treatment group, respectively;

FIG. 3 is a graph illustrating that chebulagic acid, which is one of compounds with a DP or HHDP structure, prevents onset of collagen-induced arthritis (an animal model of rheumatoid arthritis), in which , ▪ and ▴ represent saline treatment group, 10 mg/kg of chebulagic acid treatment group, and 20 mg/kg of chebulagic acid treatment group, respectively;

FIG. 4 is a graph illustrating that chebulagic acid, which is one of compounds with a DP or HHDP structure, suppresses the progression of collagen-induced arthritis (an animal model of rheumatoid arthritis), in which and ▴ represent saline treatment group and 20 mg/kg of chebulagic acid treatment group, respectively;

FIG. 5 is a graph illustrating that chebulagic acid, which is one of compounds with a DP or HHDP structure, causes a decrease in IgE within blood in an animal model with ovalbumin-induced asthma, in which Normal, OVA-Control and OVA-CHE represent a normal mice group, an asthma mice control group, and an asthma mice group treated with 20 mg/kg of chebulagic acid, respectively; and

FIG. 6 is a graph illustrating that chebulagic acid, which is one of compounds with a DP or HHDP structure, increases a success ratio and a success period of tissue transplantation/graft in an animal model with a skin allograft, in which ♦ and represent a control group and a treatment group with 20 mg/kg of chebulagic acid, respectively.

The present invention will now be described more in detail with reference to the accompanying examples, in which exemplary embodiments of the present invention are shown. It will be apparent to those skilled in the art that the present invention should not be construed as being limited to the examples set forth herein.

### EXAMPLES

In the exemplary embodiments, the animal model includes approximately 10 subjects within the same group. Except for the animal model, at least more than 3 samples/specimens are set within the same group. Experimental results are analyzed via Student's t-test. A statistic significance is determined when a p-value is less than approximately 0.05 (i.e., p<0.05). A symbol ‘*’ in the drawings means that confidence level of the experimental data is greater than approximately 95%. Each of the experimental data is expressed with average i standard error.

### Preparation Example 1

### Extraction, Purification and Analysis of Chebulagic Acid, Punicalagin and Corilagin

For chebulagic acid, approximately 500 g of immature seeds of dried Terminalia chebula were prepared in powders by a grinder. Then, approximately 4 L of 70% acetone was added to the powders and agitated at a room temperature for approximately 2 hours, thereby obtaining an extract. After the extraction, filtration, evaporation, and lyophilization were performed to obtain an extracted solid substance, which was subsequently dissolved with approximately 1 L of water. Approximately 1 L of ethyl acetate was also added to the dissolved solution to obtain extracts more than three times. The extracted ethyl acetate layer was prepared again as the solid substance through the aforementioned evaporation operation, and the solid substance was dissolved with approximately 50% methanol and then filtered. Afterwards, the filtered solution was put into a C-18 reverse-phase high performance liquid chromatography (HPLC) column, whose size was defined by approximately 30 mm×approximately 250 mm (Shimadzu, Japan). A solution of 30% methanol containing 0.05% trifluoroacetic acid (TFA) was used to elute an effective component. A peak of the effective component was subjected again to the evaporation operation to solidify the effective component, dissolved with 50% methanol and then filtered. The filtered solution passed through a C-18 reverse-phase HPLC column, whose size was defined by approximately 20 mm×approximately 250 mm (Shimadzu, Japan) to purify the peak of the effective component. Approximately 2 g of chebulagic acid, which was the finally purified effective component, was verified as the known chebulagic acid by using ZQ-mass spectrometery (MS) (Waters, USA) and AMX 500 MHz nuclear magnetic resonance (NMR) (Bruker, USA) It was also verified that the purification level of the obtained chebulagic acid was greater than approximately 98%.

For punicalagin, approximately 500 g of pericarps of dried Punica granatum were prepared in powders by a grinder. Then, approximately 4 L of 70% acetone was added to the powders and agitated at room temperature for approximately 2 hours, thereby obtaining an extract. After the extraction, filtration, evaporation, and lyophilization were performed to obtain an extracted solid substance, which was subsequently dissolved with approximately 1 L of water. The dissolved solution was put into a HP-20 column whose size was defined by approximately 50 mm×approximately 1,000 mm (Sam-Yang, Korea), and a solution of 30% methanol was used to elute an effective component. A peak of the effective component was subjected again to the evaporation operation to solidify the effective component, dissolved with 50% methanol and then filtered thereafter. The filtered solution passed through a C-18 reverse-phase HPLC column whose size was defined by approximately 20 mm×approximately 250 mm (Shimadzu, Japan) to purify the peak of the effective component. Approximately 4 g of punicalagin, which was the finally purified effective component, was verified as the known punicalagin by using ZQ-mass spectrometery (MS) (Waters, USA) and AMX 500 MHz nuclear magnetic resonance (NMR) (Bruker, USA). It was also verified that the purification level of the obtained punicalagin was greater than approximately 98%.

For corilagin, approximately 200 g of leaves of Acer nikoense were extracted by using hot water. The extract was solidified through evaporation and lyophilization operation and dissolved with 50% methanol. The dissolved solution was then filtered and passed through a C-18 reverse-phase HPLC column whose size was defined by approximately 20 mm×approximately 250 mm (Shimadzu, Japan) to purify a peak of an effective component. Approximately 5 g of corilagin, which was the finally purified effective component, was verified as the known corilagin by using ZQ-mass spectrometery (MS) (Waters, USA) and AMX 500 MHz nuclear magnetic resonance (NMR) (Bruker, USA). It was also verified that the purification level of the obtained corilagin was greater than approximately 98%.

### Example 1

### Effects on the Induction of Regulatory T Cell

An expression level of Foxp3, which is a specific transcription factor of regulatory T cell, was measured using a real-time reverse transcription—polymerase chain reaction (RT-PCR) to verify that compounds with a DP structure, especially a HHDP structure, specifically augment the regulatory T cell activity.

CD4⁺ T cells were obtained from the spleens of mice, and each group of the CD4⁺ Tcells with a concentration of approximately 1×10⁶ ml⁻¹ was suspended in Iscove's modified Dulbecco's medium (IMDM) supplemented with 10% fetal bovine serum (Gibco, USA). The CD4⁺ T cells were stimulated by antibodies of anti-CD3 (BD Bioscience, USA) and anti-CD28 (BD Bioscience, USA) and cultured at a cell incubator maintained with 5% CO₂ at 37° C. The entire RNA was extracted using RNA-Bee solution (Tel-test, USA) and then reverse transcribed into cDNA through the known method as described by McIntyre et al. (Arthritis Rheum., 48(9), pp. 2652-2659, 2003). Afterwards, the level of mRNA expression was estimated by a real-time quantitative PCR with SYBR Green I (Roche, USA). Each reaction was run in triplicate using SYBR Green PCR Master Mix (Roche, USA) according to the manufacturer's protocol. The primer base sequences used in these reactions were the sequence number 1 to the sequence number 4. At this time, the PCR was performed sequentially at 94° C. for 30 seconds, at 58° C. for 30 seconds, and at 72° C. for 30 seconds. This cycle of PCR was repeated 40 times.

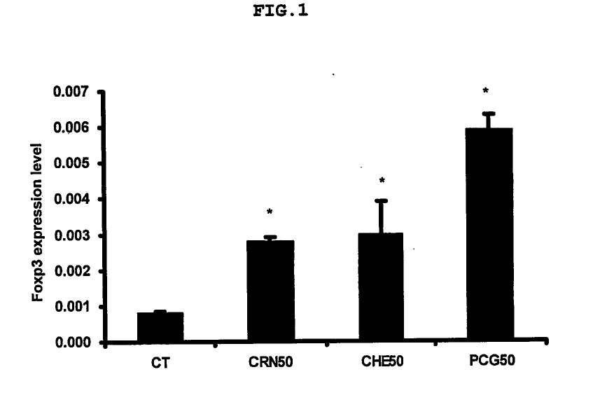

FIG. 1 shows the level of Foxp3 expression. Each of corilagin CRN, chebulagic acid CHE and punicalagin PCG had a concentration of approximately 50 μM. Each of the experimental data was expressed as average±standard error with a P* value of less than 0.05.

As illustrated in FIG. 1, the above three compounds with the DP structure, preferably the HHDP structure, increased expression of Foxp3, which is a specific transcription factor of regulatory T cells, at the concentration of approximately 50 μM. Compared with the control group CT, the increased amounts of corilagin, chebulagic acid and punicalagin were approximately 350%, approximately 375%, and approximately 737%, respectively. Based on the experimental result, it was verified that the three compounds with the DP or HHDP structure increased the regulatory T cell activity remarkably.

### Example 2

### Suppressive Effects on the Immune Response Between Allogenic Human Leukocytes

A mixed leukocyte reaction (MLR) was tested to check whether compounds with a DP structure, preferably a HHDP structure, had efficacy on the immune response between allogenic human leukocytes, as seen in such immune responses including a transplantation rejection response and a graft-versus-host disease.

Leukocytes were separated from peripheral blood of two allogenic volunteers, and then suspended with a concentration of approximately 2×10⁶ ml⁻¹ in IMDM supplemented with 10% fetal bovine serum (Gibco, USA). 100 μl of each cell suspension was added and mixed in the each well of 96-well cell culture plate. Afterwards, saline solution was added to a control group, while chebulagic acid or punicalagin, which was diluted with saline solution by 2-fold, was added to treatment groups. Then, the plates were incubated with 5% CO₂ at 37° C. for 5 days. 20 μl (approximately 0.5 μCi) of tritiated thymidine (³H-TdR) was added to each well and incubated for 8 hours.

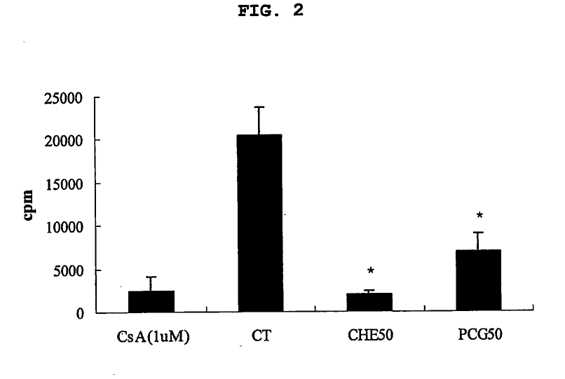

After the incubation, the leukocytes at each well were adsorbed on a glass fiber filter by a cell harvester (Cambridge, USA) and dried thereafter. Using Scintillation beta counter (Packard, USA), an amount of ³H-TdR incorporated within the cells was measured in a cpm value. This measurement indicates the level of lymphocyte proliferation, which-is illustrated in FIG. 2.

In FIG. 2, CsA represents a group with 1 μM of cyclosporin A used as a positive standard specimen. The concentration of each chebulagic acid and punicalagin was approximately 50 μM. Each of the experimental data was expressed as average±standard error with a p* value of less than 0.05.

As illustrated in FIG. 2, chebulagic acid and punicalagin with a DP or HHDP structure showed significant suppression on the proliferation of lymphocytes caused by recognition of allogenic leukocytes. The cpm of chebulagic acid-treated group was approximately 9.7% of the control group, while the cpm of punicalagin-treated group was approximately 34.1% of the control group. In particular, chebulagic acid was as effective as 1 μM cyclosporin, at the concentration of approximately 50 μM.

### Example 3

### Immune Regulatory Effects in an Animal Model of Autoimmune Disease

As introduced in an article by Luross and Williams (Immunology, 103(4), pp. 407-416, 2001), a mice model with collagen-induced arthritis (CIA) was used to check whether compounds with a DP or HHDP structure had immune regulatory effects on the chronic inflammation or autoimmune diseases. Male DBA/1J mice, which were 7 to 9 weeks old (SLC Co., Japan), received a subcutaneous injection at the base of tails with approximately 200 μg of bovine type II collagen (CII) in complete Freund's adjuvant (CFA) on days 0 and 21 to induce systemic autoimmune arthritis. When the mice did not receive any treatment, signs of collagen-induced arthritis could be seen approximately 63 days on the average from the starting date.

The following two dosing models were performed to check, among compounds with a DP or HHDP structure, whether chebulagic acid was prophylactically and therapeutically effective for autoimmune arthritis (Leung et al., J. Immunol., 170, pp. 1524-1530, 2003).

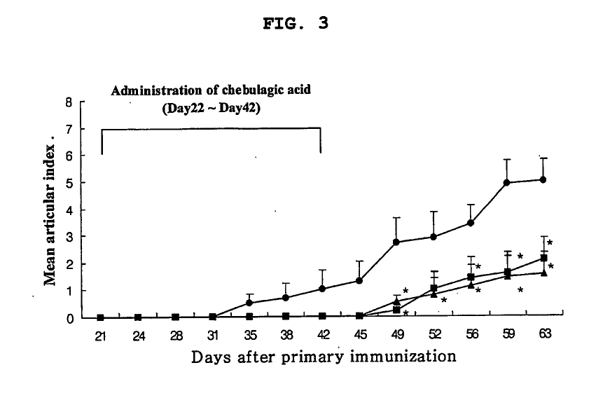

For the prophylactic dosing model, the control group and the treatment groups respectively received an intraperitoneal injection of saline solution and 10 mg/kg and 20 mg/kg of chebulagic acid daily for 3 weeks (from day 22 to day 42). A degree of arthritis of the mice was measured for every interval of 3 to 4 days from day 22 to day 63. FIG. 3 illustrates the measurement result. Particularly, FIG. 3 shows the control group with saline solution () and the treatment groups with 10 mg/kg of chebulagic acid (▪) and 20 mg/kg of chebulagic acid (▴) The number of mice for each group was 10. Each of the experimental data was expressed as average±standard error with a P* value of less than 0.05.

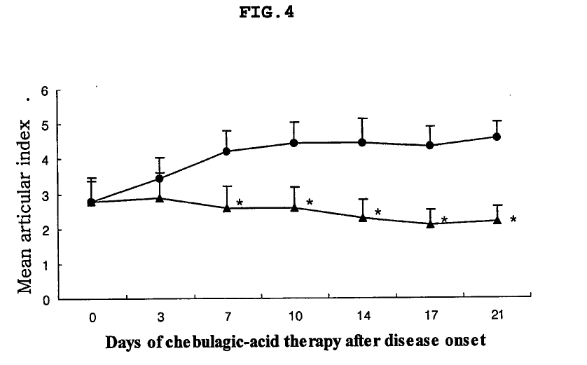

For the therapeutic doing model, another experiment was performed separately from the above experiment for the prophylactic dosing model. After all mice were induced with arthritis after day 63, the mice were divided into two groups (n=10), and the control group and the treatment group respectively received an intraperitoneal injection of saline solution and 20 mg/kg of chebulagic acid daily for 3 weeks. During 3 weeks of the treatment period, a degree of arthritis of the mice was measured for every interval of 3 to 4 days. FIG. 4 illustrates the experimental result. Particularly, FIG. 4 shows the control group with saline solution () and the treatment group with 20 mg/kg of chebulagic acid (▴). The number of mice for each group was 10. Each of the experimental data was expressed as average±standard error with a P* value of less than 0.05.

The daily observed arthritis degree of mice was scored for each paw as follows: 0=normal, 1=slight erythema and edema, 2=increased edema with loss of landmarks, 3=severe edema, and 4=severe edema with ankylosis on flexion. Each mouse was assigned an arthritic score (articular index) that equaled the sum of the scores for each paw. Each group was then assigned an arthritic score expressed as a mean articular index.

As illustrated in FIG. 3, with statistical significance, chebulagic acid with a DP or HHDP structure decreased the onset of collagen-induced arthritis and the articular index. Even though arthritis was already induced, a significant inhibition of the arthritis progression was apparent within 1 week of therapeutic administration (see FIG. 4).

### Example 4

### Effects on the Increase of Regulatory T Cells (Treg) in Vivo

A correlation between immune regulation by chebulagic acid and regulatory T cells was studied through estimation of an increase/decrease in the number of regulatory T cells in inflamed synovial tissues of the mice with collagen-induced arthritis set in specific groups in Example 3.

The knee joints of the limbs with edema were isolated and the surrounding muscle, patellar ligament and patella were removed to obtain the synovial tissues. Each of the synovial tissues was cut into small pieces and incubated twice in a buffer solution at 37° C. for 30 minutes. The buffer solution includes approximately 1 μg/ml collagenase type VI (Sigma, USA), 2% fetal bovine serum (Gibco, USA) and 1 mM EDTA (Sigma, USA).

The cells of the isolated synovial tissues were collected and stained with anti-CD4-FITC antibodies (BD Bioscience, USA) and anti-CD25-PE antibodies (BD Bioscience, USA) for fluorescence-activated cell sorter (FACS) analysis (BD Bioscience, USA). Table 1 shows a ratio of CD4⁺CD25⁺ regulatory T cells found in each group and a level of Foxp3 expression in vivo based on the method described in the first embodiment of the present invention.

TABLE 1

| | CD4⁺CD25⁺ T cell | Foxp3 expression |
|---|---|---|
| Group | ratio (%) | level |
| Normal mice group | 1.9 ± 0.2 | 1.0 ± 0.1 |
| without arthritis | | |
| Control group with | 2.5 ± 0.5 | 0.6 ± 0.2 |
| Arthritis + saline | | |
| solution | | |
| Treatment group | 5.0 ± 0.4* | 3.3 ± 0.3* |
| with arthritis + | | |
| chebulagic acid | | |
| (20 mg/kg) | | |

As shown in Table 1, the chebulagic acid treatment at a concentration of 20 mg/kg in vivo resulted in an increase of the CD4⁺CD25⁺Foxp3⁺ T cells, which are known as the regulatory T cells. Compared with the control group, the treatment group exhibited an increase of the CD4⁺CD25⁺ T cells by approximately 100% and an increase of the Foxp3 expression by approximately 550%. This experimental result demonstrated that the immune regulation by chebulagic acid with a DP or HHDP structure is accompanied with the increase of regulatory T cells generally known to be involved in the immune regulation in vivo.

### Example 5

### Inmune Regulatory Effects in an Animal Model of Hypersensitive Inflammatory Disease

The immune regulation effects by compounds with a DP structure, preferably a HHDP structure, were estimated through a mice model with ovalbumin-induced asthma (OIA). The mice model was revealed in an article by Gordon et al., J. Immunol., 175(3), pp. 1516-1522, 2005.

Balb/c mice (Samtaco, Korea) received an intraperitoneal injection of ovalbumin on days 0 and 14, and a solution of 1% ovalbumin was sprayed daily for 20 minutes on days 30, 32 and 34. 20 mg/kg of chebulagic acid was daily injected intraperitoneally for two weeks and bloods were taken to estimate the IgE level to check whether chebulagic acid had efficacy for the mice with the hypersensitive inflammatory disease.

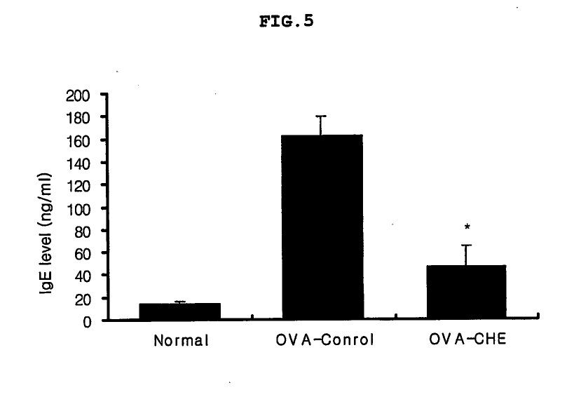

As illustrated in FIG. 5, compared with the control group OVA-Control, the treatment of chebulagic acid (20 mg/kg, OVA-CHE) in vivo decreased the IgE level closely to the IgE level estimated in the normal mice group. The IgE level is a direct indicator of the onset of a hypersensitive inflammatory disease. More specifically, the IgE level in the treatment group OVA-CHE was decreased to approximately 28.5% as compared with the control group OVA-Control. This experimental result verified that the immune regulation by chebulagic acid with a DP or HHDP structure was effective in treating hypersensitive inflammatory diseases.

### Example 6

### Immune Regulatory Effects in an Animal Model of Tissue Transplantion

To verify that compounds with a DP or HHDP structure are capable of suppressing a transplantation rejection response or a graft-versus-host disease directly in vivo, a skin graft was performed between allogenic mice and then chebulagic acid was treated to estimate the level of suppression against the tissue transplantation (Schwoebel et al., Lab Anim., 39(2), pp. 209-214, 2005).

Skin tissues including the derma (e.g., approximately 1 cm×approximately 1 cm) from the back of a Balb/c mouse (Samtaco, Korea) and a C57B1/6 mouse (Samtaco, Korea) were isolated while being anesthetized. The skin tissue of the Balb/c mouse was grafted to the back of the C57B1/6 mouse. After the next day of the skin graft, the treatment group and the control group received the daily intraperitoneal injection of 20 mg/kg of chebulagic acid and saline solution, respectively. It was checked whether the grafted skin tissues in the control group and the treatment group were normal or necrotized.

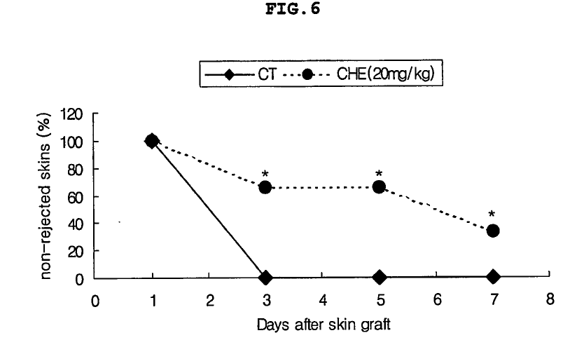

As illustrated in FIG. 6, in comparison with the control group, chebulagic acid with a DP or HHDP structure increased the success ratio and period of maintaining the tissue graft. This experimental result verified that the immune suppression between allogenic human leukocytes according to the second embodiment was consistent with the effect of suppressing the rejection response against the tissue transplantation in vivo.

### INDUSTRIAL APPLICABILITY

An immune regulatory composition increases the number and activity of regulatory T cells in vivo. Hence, the composition can be effectively used as a prophylactic and therapeutic tool against transplantation rejection, graft-versus-host diseases, autoimmune diseases, and hypersensitive inflammatory diseases.

Although the preferred embodiments of the invention have been disclosed for illustrative purposes, those skilled in the art will appreciate that various modifications, additions and substitutions are possible, without departing from the scope and spirit of the invention as disclosed in the accompanying claims.

1. A pharmaceutical composition for preventing or treating an immune disease, comprising a therapeutically effective amount of a compound with a diphenoyl (DP) structure represented by formula 1 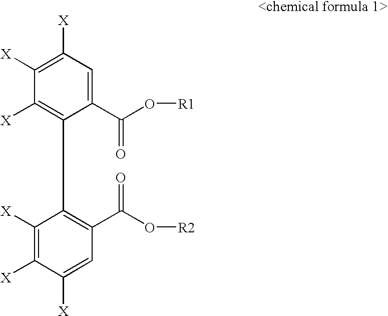
wherein, X is independently selected from the group consisting of hydrogen (H), hydroxy (OH), halogen, cyano (CN), nitro (NO), amine (NH₂), sulfonyl (SO₂), methyl (CH₃), low-grade alkoxy, and low-grade alkyl, and the halogen is selected from the group consisting of fluorine (F), chlorine (Cl), bromine (Br), and iodine (I);
R1 and R2 or O—R1 and O—R2 are independently selected from the functional group consisting of H, OH, halogen, CN, NO, NH₂, SO₂, CH₃, alkoxy, alkyl, alkenyl, alkinyl, aryl, heterocycle, and cycloalkyl, and wern the halogen is selected from the group consisting of F, Cl, Br, and I; and the compound of formula 1 may simultaneously bind with a saccharide compound or heterocyclic group at the R1 and R2 sites and may make covalent bonds with amino acids, peptides, proteins, and nucleic acids at the R1 and R2 sites.

2. The pharmaceutical composition of claim 1, wherein the compound comprises a hexahydroxydiphenoyl (HHDP) structure represented by formula 2 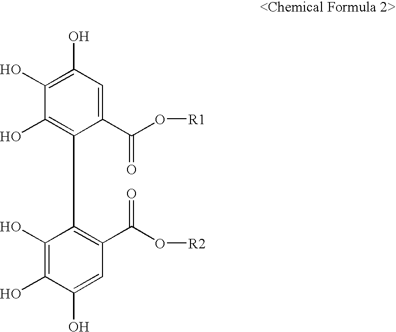
wherein R1 and R2 or O—R1 and O—R2 are independently selected from the group consisting of H, OH, halogen, CN, NO, NH₂, SO₂, CH₃, alkoxy, alkyl, alkenyl, alkinyl, aryl, heterocycle, and cycloalkyl, and the halogen is selected from the group consisting of F, Cl, Br, and I; and
the compound of formula 2 may simultaneously bind with a saccharide compound or heterocyclic group at the R1 and R2 sites and may make covalent bonds with amino acids, peptides, proteins, and nucleic acids at the R1 and R2 sites.

3. The pharmaceutical composition of claim 2, wherein the compound is selected from the group consisting of chebulagic acid, punicalagin, corilagin, and pedunculagin.

4. The pharmaceutical composition of claim 3, wherein the compound is chebulagic acid.

5. (canceled)

6. (canceled)

7. The pharmaceutical composition of claim 2, wherein the compound is obtained in the form of extract from a plant.

8. The pharmaceutical composition of claim 7, wherein the compound comprises chebulagic acid in the form of extract from Terminalia chebula or Erodium stephanianum Willd.

9. The pharmaceutical composition of claim 7, wherein the compound comprises punicalagin in the form of extract from Punica granatum.

10. The pharmaceutical composition of claim 7, wherein the compound comprises corilagin in the form of an extract from Acer nikoense.

11. (canceled)

12. A proliferation accelerating agent of regulatory T cells involved in an immune-regulatory function, comprising a compound of formula 1 defined in claim 1.

13. A method for preventing or treating an immune disease, comprising administering to a subject a compound of formula 1 defined in claim 1.

14. (canceled)

15. The pharmaceutical composition of claim 1, wherein the immune disease is selected from the group consisting of transplant rejection response, graft-versus-host disease, autoimmune disease, and hypersensitive inflammatory disease.

16. The pharmaceutical composition of claim 15, wherein the autoimmune disease is selected from the group consisting of rheumatoid arthritis, psoriasis, inflammatory bowel disease, diabetes mellitus, ulcerative colitis, multiple sclerosis, dermatosclerosis, myasthenia gravis, polymyositis, dermatomyositis, autoimmune hemolytic anemia, vasculitis syndrome, and systemic erythematosus lupus.

17. The pharmaceutical composition of claim 15, wherein the hypersensitive inflammatory disease is asthma or allergies.

18. The pharmaceutical composition of claim 7, wherein the immune disease is selected from the group consisting of transplant rejection response, graft-versus-host disease, autoimmune disease, and hypersensitive inflammatory disease.

19. The pharmaceutical composition of claim 18, wherein the autoimmune disease is selected from the group consisting of rheumatoid arthritis, psoriasis, inflammatory bowel disease, diabetes mellitus, ulcerative colitis, multiple sclerosis, dermatosclerosis, myasthenia gravis, polymyositis, dermatomyositis, autoimmune hemolytic anemia, vasculitis syndrome, and systemic erythematosus lupus.

20. The pharmaceutical composition of claim 18, wherein the hypersensitive inflammatory disease is asthma or allergies.

21. A proliferation accelerating agent of regulatory T cells involved in an immune-regulatory function, comprising a compound of formula 2 defined in claim 2.

22. The proliferation accelerating agent of claim 21, wherein the compound is chebulagic acid, punicalagin, corilagin, or pedunculagin.

23. A method for preventing or treating an immune disease, comprising administering to a subject a compound of formula 2 defined in claim 2.

24. The method of claim 23, wherein the compound is chebulagic acid, punicalagin, corilagin, or pedunculagin.

25. The method of claim 23, wherein the immune disease is selected from the group consisting of transplant rejection response, graft-versus-host disease, autoimmune disease, and hypersensitive inflammatory disease.


SEQUENCE LISTING (4 sequences)

SEQ ID NO: 1 - DNA, 23 bp, Artificial Sequence
  forward primer for murine Foxp3
aagcctatggctccttccttggc

SEQ ID NO: 2 - DNA, 24 bp, Artificial Sequence
  reverse primer for murine Foxp3
tgactaggggcactgtaggcagct

SEQ ID NO: 3 - DNA, 25 bp, Artificial Sequence
  forward primer for murine actin
tcacccacactgtgcccatctacga

SEQ ID NO: 4 - DNA, 25 bp, Artificial Sequence
  reverse primer for murine actin
cagcggaaccgctcattgccaatgg